(19) United States
(12) Patent Application Publication
(10) Pub. No.: US 2023/0381691 A1
(43) Pub. Date: Nov. 30, 2023

(54) FILTER DEVICE AND SEPARATING APPARATUS

(71) Applicant: FAUDI Aviation GmbH, Stadtallendorf (DE)

(72) Inventor: Uwe Lauber, Linden/Leihgestern (DE)

(21) Appl. No.: 18/031,390

(22) PCT Filed: Oct. 13, 2021
(86) PCT No.: PCT/EP2021/078347

§ 371 Date: Apr. 12, 2023

(30) Foreign Application Priority Data
Oct. 13, 2020 (EP) 20201624.2

(51) Int. Cl. B01D 29/23; B01D 17/04; B01D 36/00; C07C 7/144

(52) U.S. Cl. CPC B01D 29/232; B01D 17/045; B01D 36/003; C07C 7/144; B01D 2201/0407; B01D 2201/0415; B01D 2201/0461

(57) ABSTRACT

A filter device has a support tube, which has an inner face and an outer face and which forms an interior adjoining the inner face, into which interior an inlet opening leads, the support tube having through-openings, which connect the interior to a surrounding space adjoining the outer face of the support tube, and a first filter element, which has an inner face and an outer face. The first filter element is disposed in the interior of the support tube and the outer face of the first filter element is adjacent to the inner face of the support tube. A support body is disposed in the interior of the support tube and the support body adjoins the inner face of the first filter element. The filter device can be disposed in a filter housing and, in particular, can also be used in separating water from hydrocarbons.

The invention relates to a filter device according to the preamble of claim 1, a separating apparatus as claimed in claim 14, and the use of a filter device as claimed in claim 15.

Filter devices serve for holding back unwanted substances from a fluid. Thus, for example, coalescence separators are known, which serve for separating one medium from a medium being purified. For this, they have as their filter element a material which is less passable by the medium being separated than the medium being purified. In this way, increasingly larger drops of the medium being separated form inside the filter element, which rise or fall as soon as they reach the exit side of the filter element on account of their size and a density difference compared to the medium being purified and which can be collected in a given collection chamber. This collection chamber can then be emptied.

Thus, for example, it is known from DE 38 18 595 A1 how to make a coalescence separator in that a perforated support tube forms a winding core for a fiberglass-reinforced polyamide mat, so that the polyamide mat wound in multiple layers on the support tube forms a filter element. The support tube is then closed at its ends by a first end disk, forming an inlet, and a second sealing end disk. The coalescence separator is then used to introduce hydrocarbon containing free water through the inlet into the support tube and to deliver it from the inside to the outside through the filter element. The free water is held back in this process and forms drops, which drain down once they reach the outer surface of the filter element.

A similar winding of a support core with a matlike fiberglass material to form a filter device or a coalescence separator is also described in DE 21 26 080 C3. Here, there is additionally arranged inside the support tube a folded filter, receiving a flow from the inside to the outside and filtering out solids in a pre-filter stage. The folded filter is braced against the inner face of the support tube.

In various applications of such filter devices it has been discovered that the filter performance sharply declines after a short period of use. This has been attributed to cracks in the folded filter. In experiments to analyze the crack damage it was found that the folded filter is compressed to the point of collapsing at high rates of back flow, thereby becoming irreparably damaged. Such back flow can occur in connection with the use of a switching off of the forward directed delivery flow, for example when the fluid flows backward back into a lower lying tank.

Based on this fact, the problem to be solved was therefore to prevent damage to the folded filter, in order to increase the service life of the coalescence separator. The solution should be economical and reliable, and also not reduce the efficiency of the coalescence separator or the filter device in the normal flow direction if at all possible.

The main features of the invention are indicated in the characterizing passage of claim 1 and claims 14 and 15. Embodiments are the subject matter of claims 2 to 13 as well as the description.

The invention relates to a filter device, having a support tube, which has an inner face and an outer face, forming on the inner face an interior space into which an inlet opening empties, and having through openings, which connect the interior space to a surrounding space situated on the outer face of the support tube. The filter device furthermore comprises a first filter element with an inner face and an outer face, the first filter element being arranged in the interior space of the support tube with its outer face situated adjacent to the inner face of the support tube. It is proposed that a support body is arranged in the interior space of the support tube and adjacent to the inner face of the first filter element.

What is advantageous about this is that the first filter element can be braced against the support body in the event of a back flow. In this way, collapsing and/or crushing of the first filter element during a back flow is prevented.

According to a more specific embodiment of the filter device, it is proposed that the support body extends along the support tube, in particular, also along the first filter element. In this way, the support body can evenly support the first filter element.

In one special embodiment, the first filter element is a folded filter. Such folded filters have a rather good natural stability, but after a limit value is passed for the back flow volume flow they fail relatively suddenly, usually including cracks in the region of the folds. The support body can prevent this in reliable manner. Such folded filters have folds. These may wind along the support tube or be oriented transversely to the support tube like a bellows. Especially preferably, however, the folds extend along the support tube, especially in a linear manner. Folds of the latter kind in particular result in less natural stability during transverse loading, as in the back flow, so that the support body represents an effective means of bracing.

Optionally, it is proposed that the support body has the shape of a helix, a spiral, or a screw. This can be produced easily and economically, provides homogeneously distributed supporting points, has a good natural stability with low weight and material input, and allows a flow through the interior space in the longitudinal direction. A helix is a curve winding along the envelope surface of a cylinder at constant pitch.

In particular, a pitch angle of the support body can be greater than 20 degrees, preferably greater than 25 degrees. In this way, a large portion of the inner face of the first filter element is kept clear for the flow. This can also be favored by an embodiment whereby a pitch height of the support body amounts to at least 50%, preferably at least 70% of the overall diameter of the support body.

In one cost-favorable variant, the support body is formed at the inlet. This is usually sufficiently structurally stable for the supporting and can be produced especially economically.

According to one possible embodiment, the support body is a helical spring, especially a helical compression spring. Such a spring can be especially easily installed and is elastically deformed back to its original shape after the installation or under back flow stresses. At the same time, it can become deformed under back flow and thereby reduce the peak loads at the supporting points of the first filter element.

Optionally, the support body can consist for example of plastic and/or metal.

Furthermore, the filter device can be configured such that a material cross section area of the support body amounts to at most 10%, preferably at most 5% of the overall cross section area of the support body. This results in a slender support body with low material input, having the least possible effect on the fluid flows in the interior space of the support tube.

Good stability with low material input can also be achieved by an embodiment according to which the support body has a round material cross section area and/or overall cross section area.

The support element is especially effective in configurations where the support tube and the first filter element lie loosely against each other in a middle region and are (only) sealed against each other in the region of the two ends of the support tube. In fact, the middle region here is especially labile during a back flow and it is effectively braced with the support body.

In the region of each end of the support tube a closure element can be arranged. The inlet opening is then formed preferably in one of the closure elements.

For a multistage treatment of a fluid with the filter device, a second filter element can sit on the outer face of the support tube.

For a coalescing treatment of the fluid, at least one filter coat layer of the second filter element can be hydrophobic. In this way, for example, free water can be removed from a fuel.

Alternatively, the option exists that at least one filter coat layer of the second filter element is hydrophilic. This is suitable, for example, when separating free oil from water.

Furthermore, the filter device can optionally have a protective casing which encloses the filter element. The protective casing is preferably a hydrophilic textile, especially preferably a hydrophilic cotton stocking. The hydrophilia supports the drainage of large drops of the fluid being separated. A cotton stocking is especially economical for this.

The invention furthermore relates to a separating apparatus having a filter device as described above and in the following, and having a filter housing with an interior space, in which the filter device is arranged, wherein a housing entrance leads into the inlet opening of the filter device, wherein the interior space of the support tube is connected by the through openings to the interior space of the filter housing, and wherein a housing drain leads out from the interior space of the filter housing.

Optionally, a collection volume can be formed beneath the filter device in the filter housing, with a housing drain emerging from the filter housing above the collection volume.

Finally, the invention relates to the use of a filter device as described above and in the following, wherein a second filter element sits on the outer face of the support tube, wherein at least one filter coat layer of the second filter element is hydrophobic, when carrying a flow of fluid based on a hydrocarbon, wherein solids are filtered out from the fluid with the first filter element, and wherein free water is separated from the fluid with the second filter element.

Further features, details and benefits of the invention will emerge from the wording of the claims as well as the following description of exemplary embodiments with the aid of the drawings.

### THERE ARE SHOWN

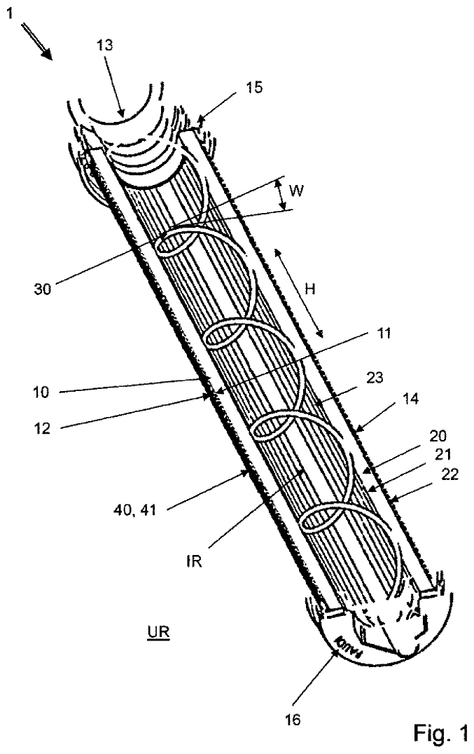

FIG. 1 a longitudinal cross section through a filter device; and

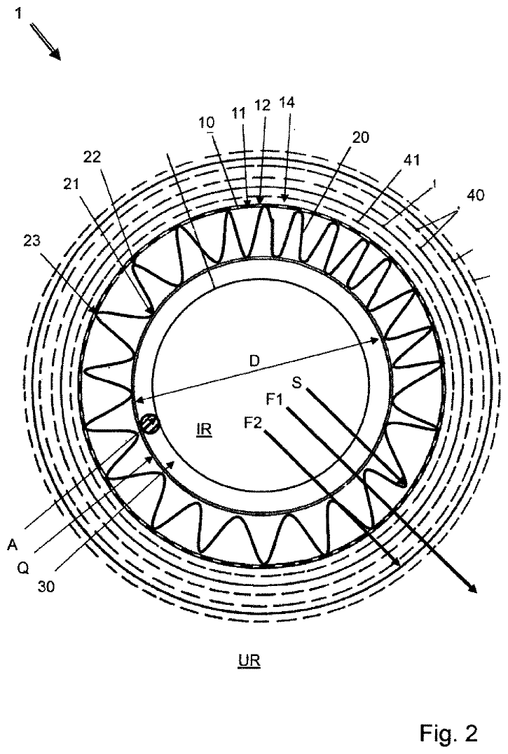

FIG. 2 a transverse cross section through a filter device.

FIG. 1 shows a longitudinal section and FIG. 2 a transverse section through a filter device 1. The same reference numbers therefore pertain to equivalent components and FIGS. 1 and 2 will be described together. The filter device 1 comprises a support tube 10 having an inner face 11 and an outer face 12, with an interior space IR formed on the inner face 11. At the end, the support tube 10 is closed by a closure element 15, 16, while an inlet opening 13 empties through one of the closure elements 15 into the interior space IR. Through openings 14 are formed in the support tube 10, connecting the interior space IR to a surrounding space UR situated at the outer face 12 of the support tube 10.

In the support tube 10 there is located a first filter element 20, especially a folded filter, having an inner face 21 and an outer face 22. The first filter element 20 is arranged with its outer face 22 adjacent to the inner face 11 of the support tube 10. Folds 23 of the first filter element 20 extend along the support tube 10.

Furthermore, a support body 30 is arranged in the interior space IR of the support tube 10 and adjacent to the inner face 21 of the first filter element 20. This extends along the support tube 10. The support body 30 here has the shape of a single-thread helix, i.e., a curve winding around the envelope surface of a cylinder with constant pitch. The pitch angle W of the helical support body 30 is greater than 20 degrees. The pitch height H of the helical support body 30 is more than 50% of the overall diameter D of the support body 30. A round material cross section area A of the support body 30 is less than 10% of the round overall cross section area Q of the support body 30. In particular, the support body 30 is a helical compression spring made of metal.

The support tube 10 and the first filter element 20 lie loosely against each other in a middle region and are only sealed off against each other in the region of the two ends of the support tube 10, especially adjacent to the closure elements 15, 16.

On the outer face 12 of the support tube 10 is located a second filter element 40 having multiple filter coat layers 41. At least one of these can be, for example, hydrophilic or hydrophobic.

If a fluid F1 consisting of a hydrocarbon flows through the filter device 1, solids S can be filtered out from the fluid F1 with the first filter element 20, as indicated in FIG. 2. Free water F2 can be separated from the fluid F1 with the second filter element 40.

The invention is not limited to one of the above-described embodiments, but instead can be modified in many ways.

All of the features and benefits emerging from the claims, the description, and the drawing, including design details, spatial arrangements, and method steps, may be essential to the invention either in themselves or in the most diverse of combinations.

### LIST OF REFERENCE NUMBERS

1 Filter device
5 Support tube
11 Inner face
12 Outer face
13 Inlet opening
14 Through opening
15 Closure element
16 Closure element
20 First filter element
21 Inner face
22 Outer face
23 Fold
30 Support body
40 Second filter element
41 Filter coat layer
A Material cross section area
D Overall diameter
F1 Fluid
F2 Free water
H Pitch height
IR Interior space
Q Overall cross section area
S Solids
UR Surrounding space
W Pitch angle

1. A filter device (1), having a support tube (10),
which has an inner face (11) and an outer face (12),
forming on the inner face (11) an interior space (IR) into which an inlet opening (13) empties, and
having through openings (14), which connect the interior space (IR) to a surrounding space (UR) situated on the outer face (12) of the support tube (10),
and having a first filter element (20) with an inner face (21) and an outer face (22), the first filter element (20) being arranged in the interior space (IR) of the support tube (10) and its outer face (22) being situated adjacent to the inner face (11) of the support tube (10),
characterized in that
a support body (30) is arranged in the interior space (IR) of the support tube (10) and adjacent to the inner face (21) of the first filter element (20).

2. The filter device (1) as claimed in claim 1, characterized in that the support body (30) extends along the support tube (10).

3. The filter device (1) as claimed in claim 1, characterized in that the first filter element (20) is a folded filter.

4. The filter device (1) as claimed in claim 1, characterized in that the support body (30) has the shape of a helix, a spiral, or a screw.

5. The filter device (1) as claimed in claim 4, characterized in that a pitch angle (W) of the support body (30) is greater than 20 degrees, preferably greater than 25 degrees.

6. The filter device (1) as claimed in claim 4, characterized in that a pitch height (H) of the support body (30) amounts to at least 50%, preferably at least 70% of the overall diameter (D) of the support body (30).

7. The filter device (1) as claimed in claim 4, characterized in that the support body (30) is formed at the inlet.

8. The filter device (1) as claimed in claim 1, characterized in that the support body (30) is a helical spring, especially a helical compression spring.

9. The filter device (1) as claimed in claim 1, characterized in that a material cross section area (A) of the support body (30) amounts to at most 10%, preferably at most 5% of the overall cross section area (Q) of the support body (30).

10. The filter device (1) as claimed in claim 1, characterized in that the support body (30) has a round material cross section area (A) and/or overall cross section area (0).

11. The filter device (1) as claimed in claim 1, characterized in that a second filter element (40) sits on the outer face (12) of the support tube (10).

12. The filter device (1) as claimed in claim 1, characterized in that at least one filter coat layer (41) of the second filter element (40) is hydrophobic.

13. The filter device (1) as claimed in claim 1, characterized in that at least one filter coat layer (41) of the second filter element (40) is hydrophilic.

14. A separating apparatus having a filter device (1) as claimed in claim 1 and having a filter housing with an interior space, in which the filter device (1) is arranged, wherein a housing entrance leads into the inlet opening (13) of the filter device (1), wherein the interior space (IR) of the support tube (10) is connected by the through openings (14) to the interior space of the filter housing, and wherein a housing drain leads out from the interior space of the filter housing.

15. The use of a filter device (1) as claimed in claim 12 when carrying a flow of fluid (F1) based on a hydrocarbon, wherein solids (S) are filtered out from the fluid (F1) with the first filter element (20), and wherein free water (F2) is separated from the fluid (F1) with the second filter element (40).
